(19) United States
(12) Patent Application Publication
(10) Pub. No.: US 2008/0241323 A1
(43) Pub. Date: Oct. 2, 2008

(54) AGRICULTURAL PRODUCT DISINFECTING SYSTEM

(76) Inventors: D. Frank Kelsey, Grand Junction, CO (US); Johnnie M. Harden, Frostproof, FL (US)

Correspondence Address: Timothy H. Van Dyke;Beusse Wolter Sanks Mora & Maire, P.A., Suite 2500, 390 North Orange Avenue, Orlando, FL, 32801, US

(21) Appl. No.: 11/695,107

(22) Filed: Apr. 2, 2007

(51) Int. Cl. A61L 2/00; A23B 7/144; A61L 2/18; A61L 2/20; A23B 7/153

(52) U.S. Cl. 426/318; 422/300; 422/304; 422/33; 422/37; 426/335

(57) ABSTRACT

Disclosed herein is a system for sanitizing objects, particularly food products such as fruits and vegetables. The system includes two or more tanks for generation and storage of chlorine dioxide. The system provides for the simultaneous use and generation of chlorine dioxide so that the system avoids undesired down-time.

### BACKGROUND

Various microorganisms cause significant financial losses due to decay as fresh produce travels through marketing and handling channels from producer to consumer. Some of the more destructive organisms include Erwinia spp bacteria and Geotrichum candidum mold, both of which are pathogenic to a wide variety of fruits and vegetables and can cause significant losses within the industry on an annual basis. These organisms are prevalent in the fields where produce is grown, and can be spread during and after harvest through subsequent handling. These organisms, and many others, can be spread during produce washing operations, thus it is desirable to apply a sanitizer to fruits and vegetables to reduce the likelihood that otherwise sound fruit may become infected with decay causing pathogens.

A conventional way of sanitizing produce comprises washing or spraying of objects, such as fruit or more particular tomatoes and citrus fruit, in conveyor systems from a fixed overhead spray manifold as the objects are being scrubbed or cleaned in a conveyor brush bed. The purpose for washing the tomatoes and citrus fruit may include, for example, not only removal of field dirt, contaminants and pesticides, but also, particularly in the case of high-pressure spraying, removal of scale insects or fungal microorganisms which might otherwise mar the appearance or cause later spoilage of the fruit. Such spray washing may occur in conjunction with treatment of fruit with special solutions, usually water soluble, as described, for example, in U.S. Pat. No. 4,990,351 (Orman, et al); U.S. Pat. No. 5,007,335 (Orman, et al); and U.S. Pat. No. 5,148,738 (Orman, et al)—all assigned to Sunkist Growers, Inc.

Typically, the fruit are loaded onto a brush bed conveyor system which aligns the fruit into the linear pockets between elongated cylindrical brush rollers, which are aligned transversely to the movement of the conveyor. A system of nozzles on a fixed manifold located above the brush bed rollers directs fluid onto the fruit passing below it. The rollers brush, scrub and rotate the fruit while the fruit is being simultaneously spray washed from above by the fixed spraying system. As described in one or more of the aforementioned patents, the solution may be filtered and recirculated for conservation and economy.

Sanitization of produce is an important issue in the agricultural industry. More efficient and less costly methods of sanitization would provide a significant benefit to the supply and distribution channels of this industry.

The predominant chemical used in the produce industry to address microbial concerns is chlorine, often in the form of sodium hypochlorite. There are many disadvantages associated with the use of chlorine. Chlorine must be used within a narrow pH range to ensure optimal biocidal activity in solution. Chlorine dissociates into either an acid or ion form in water. The acid form (hypochlorus acid) is the preferred form for biological activity. Adding liquid sodium hypochlorite to water results in an increase in solution pH, often above the narrow optimal range for produce operations of pH 6.5-7.5. This requires operators to employ additional equipment to monitor and control solution pH, thus increasing chemical inputs (in the form of an acid), costs, and complexity associated with using sodium hypochlorite. Expensive and complex sensing, feedback, and control systems are often utilized to balance both chlorine and pH levels in produce operations. High purity chlorine dioxide is an effective biocide across a broad pH range (4-10), and remains a dissolved gas that does not dissociate in water. Use of high purity chlorine dioxide allows the operator to eliminate a chemical input to control pH, and reduces equipment and chemical costs associated with systems used to maintain chlorine solution pH.

Chlorine is also known to react with organic matter to form tri-halomethanes (THMs), and it reacts with ammonia to produce chloramines. THMs are suspected carcinogens and the US EPA has set standards limiting their presence in drinking water due to potential negative health impacts. Chloramines are a primary irritant to workers and personnel in applications where chlorine is used. Due to the presence of high levels of organic matter and preharvest chemical residues associated with produce, chloramines are likely to occur in produce washing facilities that utilize chlorine. High-purity chlorine dioxide, in contrast, does not react to form THMs, and does not react with ammonia. Thus, undesirable cross reactions are minimized by the use of high purity chlorine dioxide, increasing efficiency and addressing potential hazards associated with chlorine.

Chlorine is also known to be highly corrosive to many materials. Its corrosive qualities can increase operational costs as it degrades processing equipment over time. High purity chlorine dioxide is compatible with most materials at use rates, and thus can reduce operational costs associated with using more corrosive materials long term.

### BRIEF DESCRIPTION OF THE DRAWINGS

FIG. 1 shows a sanitizing system embodiment of the subject invention

FIG. 2 shows a flow diagram of a method embodiment of the subject invention.

FIG. 3 shows an alternative system embodiment of the subject invention.

### DETAILED DESCRIPTION

The present invention provides a method for the prevention of growth of a broad spectrum of microorganisms on food products. The prevention of growth of microorganisms on food products is intended to provide a food product that is devoid of or contains minimal numbers of viable microorganisms that could cause illness in humans or animals or spoilage of the food product prior to ingestion. The prevention of growth of microorganisms on food products is intended to include but is not limited to the following mechanisms: (1) removal of attached microorganisms from the food products; (2) inhibition of attachment of microorganisms to the food products; (3) killing or inactivation of attached microorganisms on the food products; and (4) killing or inactivation of microorganisms which are not attached to the food product but which are present in liquids associated with the food products during processing; such as in chill tanks.

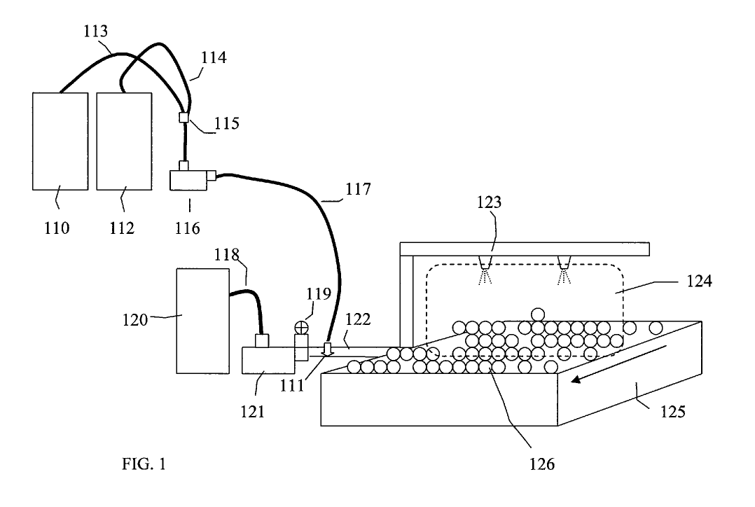

According to one embodiment, shown in FIG. 1, the subject invention pertains to a system 100 for sanitizing food product. The system 100 comprises a first generation tank 110 and a second generation tank 112 for generating and storing a concentrated sanitizing medium. In a particular embodiment, the sanitizing medium is a concentrated chlorine dioxide mixture. Each tank 110, 112 is in fluid communication with a tank fluid line 113, 114 for delivery of sanitizing medium to a chemical delivery pump 116. The fluid lines 113, 114 are conjoined to a valve 115 for selection of a given tank 110, 112 to utilize.

A water tank 120 provides a water supply to a treatment pump 121 via water supply fluid line 118. Upstream from where fluid line 118 connects to treatment pump 121 sanitizing medium is injected at injector 111 via chemical fluid line 117. Sanitizing medium is carefully injected at rate controlled by either settings on the chemical delivery pump 116 or flow rate of injector 111, or a combination thereof. The water from the water tank 120 and sanitizing medium from tanks 110 or 112 are combined to form a treatment solution. The treatment solution is delivered to a treatment staging platform 125 vie a treatment solution conduit 122, which feeds one or more nozzles 123. The treatment solution is delivered to product 126 via nozzles 123 into a treatment zone 124. The treatment pump 121 comprises a valve 119 to control pressure delivered to nozzles 123. Pressure, flow rate and droplet size may be controlled by the valve 119 and configuration of the nozzle 123. As will be discussed in further detail below, these variables can be controlled to modify the treatment regime dependent on the given needs for a product and time of product in the treatment zone 124.

The treatment staging platform shown in FIG. 1 relates to a conveyor that delivers product into and out of the treatment zone 124. In view of the teachings herein, those skilled in the art will appreciate that the treatment staging platform can take several alternate forms including, but not limited to, a conveyor, brush bed conveyor, a bin of product carried into the treatment zone, rollers, a ramp for letting product drop through the treatment zone, or a flume or other suitable platforms.

Furthermore, FIG. 1 shows a system comprising two nozzles. Those skilled in the art will appreciate that the number of nozzles implemented can be altered depending on the size of the sanitization system. In one embodiment, the number of nozzles ranges from 1-20 or more. In a more specific embodiment, the number of nozzles ranges from 3-15. Likewise, in an additional embodiment, it is contemplated that the system comprises two or more conduits each comprising a plurality of nozzles for delivery of treatment solution to a treatment zone.

The inventors have discovered that a major drawback to using a chlorine dioxide treatment solution is the down-time required to generate the chlorine dioxide. The down-time can be as large as 12 hours depending on the size of the tank. Naturally, this down-time is extremely inefficient and costly to the produce processors. The inventors have addressed this issue by designing a redundant system which comprises more than one generation tank. Accordingly, in embodiments of the subject invention, chlorine dioxide generated in one tank can be used while chlorine dioxide is being generated in a different tank. In a two tank system embodiment, the system can provide an uninterrupted supply of chlorine dioxide by rotating usage of and generation of chlorine dioxide in the tanks.

The sanitizing medium particularly useful in conjunction with the system embodiment shown in FIG. 1 is a chlorine dioxide produced via the method taught in U.S. Pat. Nos. 6,602,466 and 6,607,696 and U.S. Patent Pub. 2006/0120945. The skilled artisan will appreciate that other chemicals, fungicides, anti-microbials, etc. may be adapted for use with certain embodiments of the invention.

The efficiency of the sanitizing system may depend on a number of variables, including, but not limited to, concentration (e.g. ppm), droplet size, flow rate and pressure. The droplet size and pressure are related in that the higher the pressure the smaller the droplet size that is formed. The flow rate should be sufficient to cover the object and provide adequate treatment time dependent on how fast the object moves through the system. In one embodiment, the treatment time ranges from 5 to 60 seconds. In a more specific embodiment, the treatment time is for 15 to 30 seconds. In one embodiment, the spray droplet size is 400 microns or less. This embodiment may be particularly advantageous in closed systems, i.e. systems where the treatment zone occurs within an enclosed chamber. The smaller the droplet size, there is an increase in the propensity of out-gassing of the chlorine dioxide. In closed systems, this may be desired since the gas will stay in the chamber and remain in contact with the produce. In another embodiment, the droplet size is at least 300 microns. In a more specific embodiment, the spray droplet size ranges from 700 to 1000 microns. In a specific embodiment, the sanitizing medium comprises chlorine dioxide at a concentration of 3-10 ppm.

In another embodiment, the invention pertains to a method of sanitizing produce comprising spraying a treatment solution onto the produce; wherein the treatment solution comprises between 3-10 ppm chlorine dioxide; and wherein the spraying occurs according to a flow rate of 1-6 gallons per minute to a treatment zone comprising 8-60 square feet. In a specific embodiment, the produce is delivered through the treatment zone so as the treatment time ranges from 5-60 seconds.

The inventors have found that the efficiency of the system is sensitive and can be modulated depending on the ability to provide consistent pressure and flow rates. The flow rate and pressure of a water supply can be critical to maintain a high level of efficiency. Most water supplies from utility companies have fluctuation pressures and flow rates, which could potentially create problems for the system. The inventors have solved this problem by providing a water tank that serves a constant reservoir supply for the treatment pump.

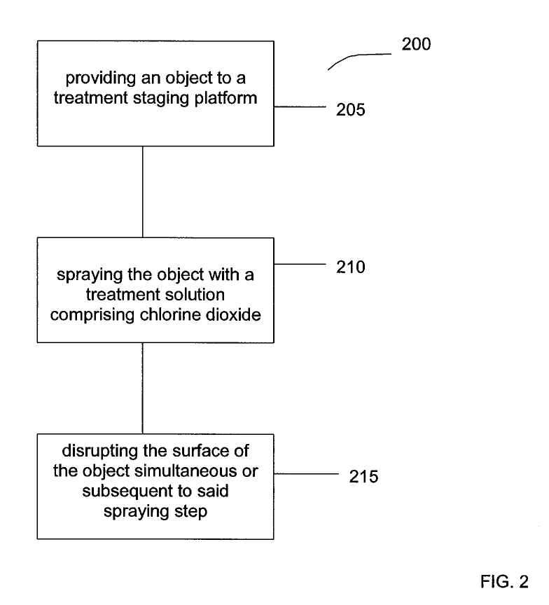

According to another embodiment, the subject invention pertains to a method of sanitizing an object as illustrated in FIG. 2. The method comprises providing an object to a treatment staging platform 205; spraying said object with a treatment solution comprising chlorine dioxide 210, wherein said concentration of treatment solution is 3-10 ppm chlorine dioxide and the droplet size is 700-1000 microns; and disrupting the surface of said object simultaneous or subsequent to said spraying step 215. The disrupting step may occur by brushes or similar disruption means that serves to aggravate microbes on the surface of the object in the presence of treatment solution. The purpose is to aggravate the microbes on the surface so as to promote contact of the treatment solution with sufficient percentage of microbes present. In an alternative embodiment, the spraying step comprises high pressure spraying which also acts to disrupt microbes on the surface of the object. According to this alternative embodiment, a separate disruption means may not be necessary. Low pressure spraying would typically be between 20-40 psi; medium high pressure spraying would fall between 40-100 psi, and high pressure spraying pertains to spraying of 100 psi or above.

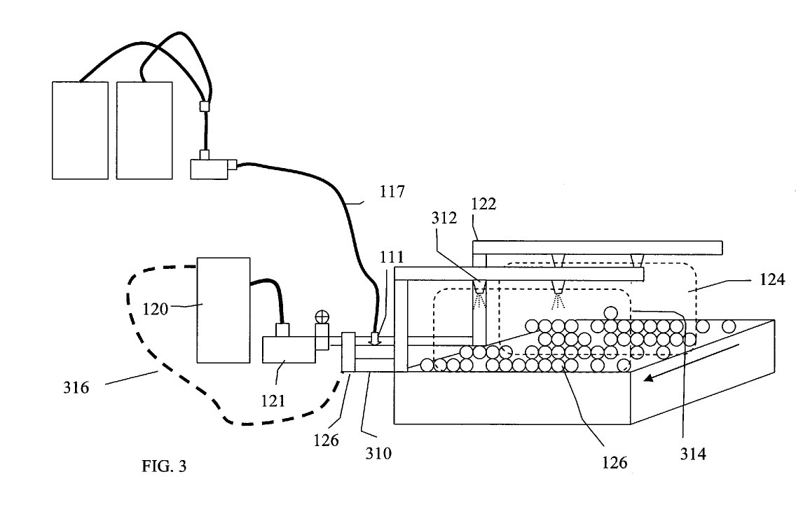

An alternative embodiment of a sanitizing system 300 is shown in FIG. 3. This system 300 is similar to the system shown in FIG. 1, and common elements are numbered the same. In this alternative system 300, sanitizing medium is delivered via line 117 and injected into conduit 122 at injector 111, similar to that shown in FIG. 1. Typically, the treatment pump 121 is capable of delivering more fluid than is needed. A shunt 126 is positioned on conduit 122 which can serve to deliver water through conduit and out of nozzles 312. This water serves as a rinse function. After product 126 is treated in the treatment zone 124, product 126 is then delivered to the rinse zone 314. Alternatively, the conduit 310 is not included in the system, and water is directed back to the water tank 120 via dashed line 316.

In yet a further embodiment, the subject invention pertains to a method of providing a treatment solution for sanitizing objects comprising obtaining a system such as that shown in FIG. 1 having more than one generation tank. The embodiment comprises a step of providing a treatment solution comprising sanitizing medium generated in a first tank, and generating sanitizing medium in a second tank during said providing step.

It is noted that the figures depicting this invention are merely representative of particular embodiments and are not meant to limit the range of possible configurations. Those skilled in the art will appreciate that the scope of this invention should be measured by the claims appended hereto, and not merely by the specific embodiments exemplified herein.

What is claimed is:

1. A system for sanitizing an object comprising
a first generation tank for generating and storing a sanitizing medium;
a second generation tank for generating and storing a sanitizing medium;
a chemical delivery pump in fluid communication with at least one of said first and second generation tanks via a tank fluid line;
a water supply;
a treatment pump in fluid communication with said water supply via a water supply line;
at least one nozzle in fluid communication with said treatment pump via a treatment solution conduit, wherein sanitizing medium is provided into said water supply line, directly into said treatment pump, or into said treatment solution conduit; and
a treatment staging platform for sanitizing an object by delivering treatment solution to a treatment zone.

2. The system of claim 1, wherein said sanitizing medium comprises chlorine dioxide.

3. The system of claim 1, wherein said water supply is a refillable water tank.

4. The system of claim 1, wherein said treatment staging platform is a brush conveyor.

5. A method of sanitizing an object comprising
spraying said object with a treatment solution comprising a concentration of chlorine dioxide of 3-10 ppm and a droplet size of at least 300 microns; and
disrupting said object simultaneous or subsequent to said spraying step.

6. The method of claim 5 further comprising obtaining a system according to claim 1 and providing said object to a treatment staging platform.

7. A method of sanitizing an object comprising
obtaining a system according to claim 1; and
spraying said object with a treatment solution for a time and pressure sufficient to sanitize said object.

8. The method of claim 7, wherein said treatment solution comprises chlorine dioxide at a concentration of 3-10 ppm.

9. The method of claim 7, wherein the treatment solution is provided in a droplet size from 700 to 1000 microns.

10. A method of sanitizing produce comprising spraying a treatment solution onto said produce; wherein the treatment solution comprises between 3-10 ppm chlorine dioxide; and wherein the spraying occurs according to a flow rate of 1-6 gallons per minute to a treatment zone comprising 8-60 square feet.

11. The method of claim 10, further comprising delivering said produce through said treatment zone so as to generate a time period in said treatment zone of 5-60 seconds.

12. The method of claim 11, wherein said time period in said treatment zone is 15-60 seconds.

13. A method of sanitizing produce comprising spraying a treatment solution onto said produce; wherein the treatment solution comprises between 3-10 ppm chlorine dioxide; and wherein the treatment solution is provided in droplets having a size of 400 microns or less.

14. The method of claim 13, wherein treatment solution is provided into a chamber comprising said produce and which retains chlorine dioxide gas released from said droplets.

15. The system of claim 1, wherein treatment solution is delivered into said treatment solution conduit and further comprising a rinse conduit in fluid communication with said treatment pump and which comprises nozzles for delivery of water to a rinse zone.
